(19) United States
(12) Patent Application Publication
(10) Pub. No.: US 2010/0057086 A1
(43) Pub. Date: Mar. 4, 2010

(54) ANODIZED LOCKING PLATE COMPONENTS

(75) Inventors: Gregory G. Price, Warsaw, IN (US); Richard C. Compton, Warsaw, IN (US)

Correspondence Address: ZIMMER TECHNOLOGY - BAKER & DANIELS, 111 EAST WAYNE STREET, SUITE 800, FORT WAYNE, IN, 46802, US

(73) Assignee: ZIMMER, INC., Warsaw, IN (US)

(21) Appl. No.: 12/549,489

(22) Filed: Aug. 28, 2009

Related U.S. Application Data
(60) Provisional application No. 61/093,195, filed on Aug. 29, 2008.

(51) Int. Cl. A61B 17/80; A61B 17/86; A61B 17/88

(52) U.S. Cl. 606/70; 606/315; 606/286; 606/281

(57) ABSTRACT

An orthopedic assembly including a plate and a locking screw that interlocks with the plate to secure the plate to a bone while the bone heals. To minimize galling or cold welding within an engagement region of the orthopedic assembly, an anodized coating, such as a Type II anodized coating, may be applied to both engaging surfaces of the engagement region. To avoid having to mask portions of the plate and the locking screw outside of the engagement region during the coating process, the anodized coating may be applied to the entire plate and the entire locking screw.

### CROSS REFERENCE TO RELATED APPLICATION

This application claims priority from U.S. Provisional Patent Application No. 61/093,195, entitled “Minimization of Galling Using Type II Anodized Locking Plate Components,” filed on Aug. 29, 2008, by the same inventors hereof, the disclosure of which is expressly incorporated herein by reference.

### BACKGROUND

1. Field of the Invention

The present invention relates to an orthopedic assembly for supporting a bone while the bone heals. More particularly, the present invention relates to an orthopedic plate and locking screws for securing the orthopedic plate to the bone while the bone heals.

2. Description of the Related Art

An orthopedic plate may be secured across a bone fracture to reduce the fracture and to provide support to the bone. The orthopedic plate may include a plurality of holes therein for receipt of screws which are drilled into the bone to secure the orthopedic plate to the bone across the fracture line.

In certain cases, the hole in the orthopedic plate may be threaded to engage the screw in a locked arrangement. Although locking the screw to the orthopedic plate provides enhanced fixation between the screw and the orthopedic plate, surgeons have encountered great difficulty when later attempting to separate the screw from the orthopedic plate. Over the last several years, researchers have published articles with potential solutions for improving the locked screw removal step. For example, researchers have proposed special extraction tools for gripping and removing stripped screws. When these solutions fail, however, the surgeon may have to cut the screw out of the orthopedic plate and the bone.

### SUMMARY

The present invention provides an orthopedic assembly including a plate and a locking screw that interlocks with the plate to secure the plate to a bone while the bone heals. To minimize galling or cold welding within an engagement region of the orthopedic assembly, an anodized coating, such as a Type II anodized coating, may be applied to both engaging surfaces of the engagement region. To avoid having to mask portions of the plate and the locking screw outside of the engagement region during the coating process, the anodized coating may be applied to the entire plate and the entire locking screw.

According to an embodiment of the present invention, an orthopedic assembly is provided and includes a screw and a plate. The screw has a distal end and a proximal end and includes a shaft, a first thread that extends from the shaft and wraps helically around the shaft to contact a bone, a head located at the proximal end of the screw that extends radially outwardly beyond the shaft, the head comprising an anodized coating, and a second thread that extends from the head and wraps helically around the head. The plate includes a bone-contacting surface configured to rest against the bone, a wall defining a bore in the plate, the bore sized to receive the screw and comprising an anodized coating, and a third thread that extends from the wall of the plate to interlock with the second thread of the screw.

According to another embodiment of the present invention, an orthopedic assembly is provided and includes a screw and a fixation device. The screw includes a first engagement surface and a threaded shaft configured to contact a bone. The fixation device includes a bone-contacting surface and a second engagement surface, the bone-contacting surface configured to contact the bone, the second engagement surface defining a bore that is sized to receive the screw such that the second engagement surface frictionally engages the first engagement surface of the screw, both the first engagement surface and the second engagement surface comprising anodized coatings.

According to yet another embodiment of the present invention, a method is provided for securing an orthopedic device to an anatomical structure. The method includes the step of providing an orthopedic assembly that includes a screw and a fixation device. The screw has a first engagement surface and a threaded shaft configured to contact a bone. The fixation device has a bone-contacting surface and a second engagement surface, the second engagement surface defining a bore. Both the first engagement surface of the screw and the second engagement surface of the fixation device include anodized coatings. The method further includes the steps of placing the bone-contacting surface of the fixation device in contact with the bone, inserting the screw through the bore of the fixation device, driving the threaded shaft of the screw into the bone, and frictionally engaging the first engagement surface of the screw with the second engagement surface of the fixation device.

### BRIEF DESCRIPTION OF THE DRAWINGS

The above-mentioned and other features and advantages of this invention, and the manner of attaining them, will become more apparent and the invention itself will be better understood by reference to the following description of embodiments of the invention taken in conjunction with the accompanying drawings, wherein:

FIG. 1 is a perspective view of an orthopedic assembly of the present invention, including a plate and multiple locking screws, secured to a femur;

FIG. 2 is a cross-sectional view of the orthopedic assembly and femur of FIG. 1, taken along line 2-2 of FIG. 1;

FIG. 3 is a plan view of the plate of FIG. 1;

FIG. 4 is an elevational view of a locking screw of FIG. 1; and

FIG. 5 is a graphical view of experimental measurements of removal torque versus seating torque for various locking screw and plate combinations.

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

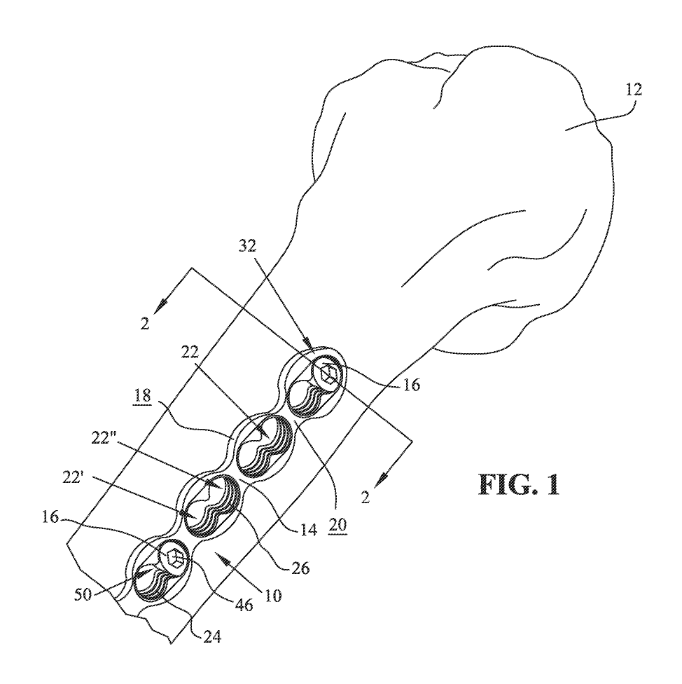

Referring to FIG. 1, orthopedic assembly 10 is shown secured to femur 12. Although orthopedic assembly 10 is described and depicted herein as being secured to femur 12, orthopedic assembly 10 may be used to support other bones of the body, such as the tibia, fibula, radius, ulna, clavicle, and any other bone. Orthopedic assembly 10 includes brace or plate 14. Although orthopedic assembly 10 is described and depicted herein as including plate 14, orthopedic assembly 10 may include other orthopedic fixation devices such as an intramedullary nail, a plate for a dynamic hip system, or a similar orthopedic fixation device. Plate 14 may be secured to femur 12 with a variety of anchors including, but not limited to, locking screws 16, cortical screws, wires, or other suitable anchors. An exemplary orthopedic assembly is the Universal Locking System generally available from Zimmer, Inc. of Warsaw, Ind.

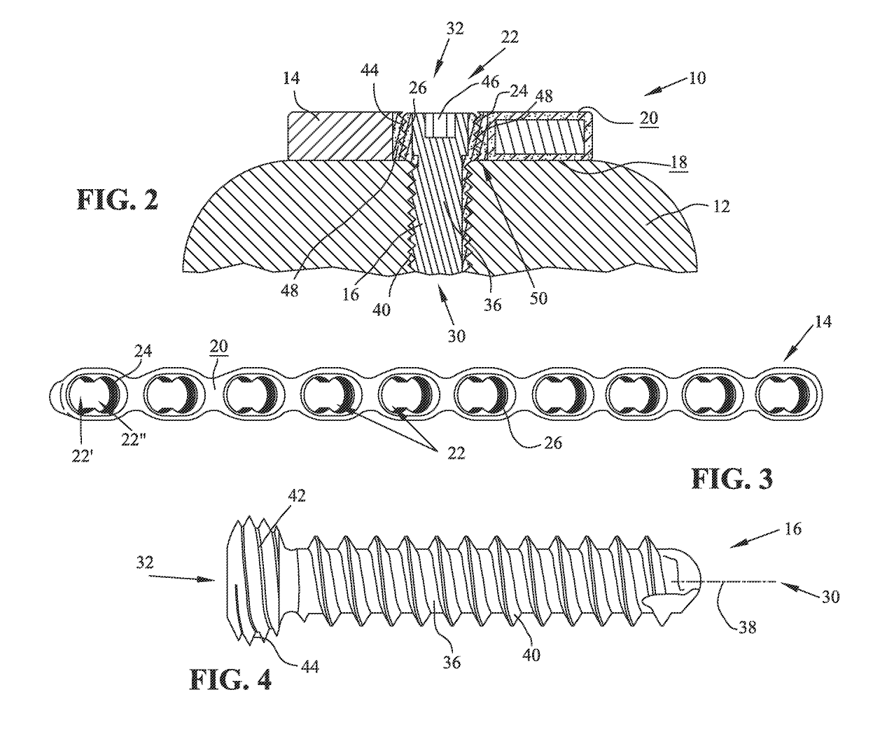

Referring to FIGS. 1-2, plate 14 includes bottom surface 18 and top surface 20. Bottom surface 18 is configured to contact or rest against femur 12. Top surface 20 is located opposite bottom surface 18 and is configured to contact soft tissue surrounding femur 12.

Plate 14 may be constructed of a rigid, biocompatible metal. For example, plate 14 may be constructed of titanium, a titanium alloy, tantalum, a tantalum alloy, zirconium, a zirconium alloy, or another suitable metal. An exemplary plate 14 may be constructed of a titanium alloy such as a Ti-15Mo alloy or a Tivanium™ mTi-6Al-4V alloy. Tivanium™ is currently generally available from and is a registered trademark of Zimmer, Inc. of Warsaw, Ind.

Plate 14 may be provided in various shapes and sizes to accommodate different bones and fractures. For example, the length of plate 14 may be small as approximately 40 millimeters (mm), 60 mm, 80 mm, or 100 mm, or as large as approximately 120 mm, 140 mm, 160 mm, 180 mm, or more.

Referring next to FIGS. 1 and 3, plate 14 further includes a plurality of bores 22 each configured to receive at least one anchor for securing plate 14 to femur 12. Depending on the size of plate 14, the number of bores 22 in plate 14 may vary. Each bore 22 extends from bottom surface 18 of plate 14 to top surface 20 of plate 14 and is defined by wall 24 of plate 14.

According to an exemplary embodiment of the present invention, wall 24 surrounding bore 22 is configured to hold locking screw 16 in frictional engagement with plate 14, as discussed below. For example, as shown in FIG. 2, wall 24 of plate 14 surrounding bore 22 may be threaded. Specifically, wall 24 of plate 14 surrounding bore 22 may include female thread 26. As another example, wall 24 of plate 14 surrounding bore 22 may include a coaxial series of annular grooves (not shown).

According to another exemplary embodiment of the present invention, each bore 22 may be configured to receive a variety of anchors in a variety of positions. Therefore, plate 14 may be configured to provide dual compression and locking of the fracture. For example, as shown in FIG. 1, bore 22 may include two overlapping holes, 22′ and 22″, defined by wall 24 having a figure-eight shape. Hole 22′ may be configured to receive a standard cortical screw, a lag screw, or a locking screw, for example. Similarly, hole 22″ may be configured to receive a standard cortical screw, a lag screw, or a locking screw, for example. Therefore, a surgeon may select a desired anchor and may choose whether to implant the selected anchor into either hole 22′ or hole 22″ of bore 22.

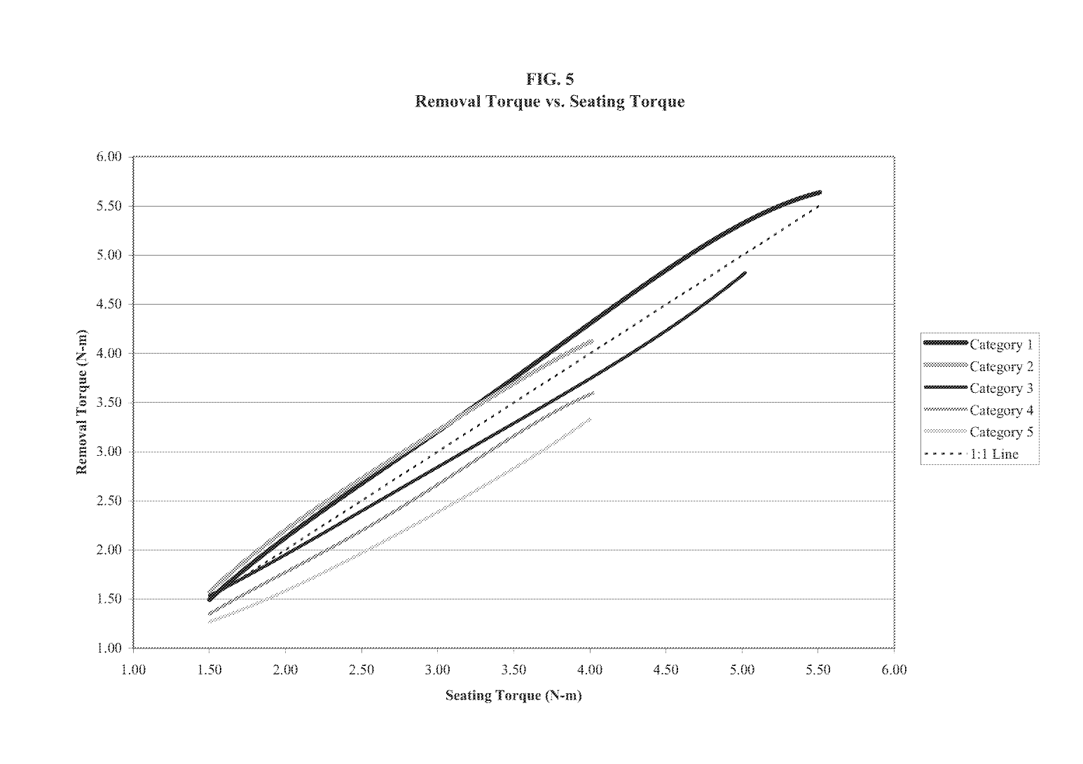

Referring to FIGS. 1 and 4, locking screw 16 includes distal end 30 and proximal end 32. As used herein, “proximal” and “distal” are determined relative to a surgeon or another user, such that distal end 30 of locking screw 16 is farther from the user than proximal end 32 of locking screw 16 during implantation. As shown in FIG. 2, proximal end 32 of locking screw 16 is located proximate plate 14 and is accessible by a surgeon, while distal end 30 of locking screw 16 extends into femur 12.

Locking screw 16 may be constructed of a rigid, biocompatible metal. For example, locking screw 16 may be constructed of titanium, a titanium alloy, tantalum, a tantalum alloy, zirconium, a zirconium alloy, or another suitable metal. An exemplary locking screw 16 may be constructed of a titanium alloy such as a Ti-15Mo alloy or a Tivanium™ Ti-6Al-4V alloy. Tivanium™ is currently generally available from and is a registered trademark of Zimmer, Inc. of Warsaw, Ind.

Referring to FIG. 4, locking screw 16 includes shaft 36 extending between distal end 30 and proximal end 32. Shaft 36 includes longitudinal axis 38 extending therethrough. Shaft 36 may taper near distal end 30 of locking screw 16 and/or include a cutting flute to facilitate self-tapping of locking screw 16 into bone.

Shaft 36 of locking screw 16 may be provided in various dimensions to accommodate various fractures and patients having bones of various dimensions. For example, the length of shaft 36 may be as small as approximately 10 mm, 20 mm, 30 mm, 40 mm, or 50 mm, or as large as approximately 60 mm, 70 mm, 80 mm, 90 mm, 100 mm, or more. As another example, the outer diameter of shaft 36 (to the root of thread 40) may be as small as approximately 1.0 mm, 1.5 mm, 2.0 mm, 2.5 mm, or 3.0 mm, or as large as approximately 3.5 mm, 4.0 mm, 4.5 mm, 5.0 mm, or more.

Shaft 36 of locking screw 16 includes thread 40 that extends helically from shaft 36 and is configured to contact bone. As shown in FIG. 2, when locking screw 16 is inserted through bore 22 of plate 14, thread 40 of locking screw 16 contacts bone of femur 12 to secure locking screw 16 to femur 12.

Referring still to FIG. 4, locking screw 16 further includes head 42 located at proximal end 32 of locking screw 16. Head 42 of locking screw 16 may extend radially outwardly beyond shaft 36.

Head 42 of locking screw 16 may include bore 46, as shown in FIGS. 1-2. Bore 46 is configured to cooperate with a tool (not shown) for driving locking screw 16 into femur 12. Bore 46 may be hexagonal, D-shaped, slotted, star-shaped, or another known shape, and configured to cooperate with a tool having a similarly shaped tip, such as a screwdriver or a wrench.

According to an exemplary embodiment of the present invention, wall 24 surrounding bore 22 is configured to hold head 42 of locking screw 16 in frictional engagement with plate 14. For example, if wall 24 of plate 14 surrounding bore 22 is threaded, head 42 of locking screw 16 may also be threaded. Specifically, if wall 24 of plate 14 surrounding bore 22 includes female thread 26, head 42 of locking screw 16 may include male thread 44 that is configured to interlock with female thread 26. As another example, if wall 24 of plate 14 surrounding bore 22 includes a coaxial series of annular grooves (not shown), head 42 of locking screw 16 may include a coaxial series of corresponding annular ridges (not shown) that are configured to create an interference fit with the annular grooves of wall 24.

Referring to FIGS. 1-2, when locking screw 16 is inserted through bore 22 of plate 14, thread 40 extending from shaft 36 of locking screw 16 contacts bone of femur 12 to secure locking screw 16 to femur 12. Additionally, male thread 44 extending from head 42 of locking screw 16 interlocks with female thread 26 on wall 24 of plate 14 to secure locking screw 16 to plate 14. Thus, a stable, locked fixation may be achieved between femur 12 and plate 14 to promote healing of femur 12.

According to an exemplary embodiment of the present invention, head 42 of locking screw 16 may be sized to fit entirely within bore 22 of plate 14. In this case, proximal end 32 of locking screw 16 may be nearly flush with plate 14 to avoid protruding into and irritating soft tissue surrounding femur 12.

According to another exemplary embodiment of the present invention, male thread 44 on head 42 of locking screw 16 may have a small pitch to maximize the number of threads captured within a narrow bore 22. Thread 40 on shaft 36 of locking screw 16 may have a larger pitch than male thread 44 on head 42 of locking screw 16, but both may be configured to advance at the same rate by extending across locking screw 16 at the same angle relative to longitudinal axis 38, as shown in FIG. 3.

Referring still to FIGS. 1-2, a stable, locked fixation between plate 14 and locking screw 16 may be desirable while femur 12 heals. After implantation, a surgeon may choose to remove orthopedic assembly 10 from femur 12, which requires that locking screws 16 be removed from femur 12 and separated from plate 14. For example, the surgeon may choose to remove orthopedic assembly 10 from femur 12 after femur 12 heals or if complications arise. However, over time, galling or cold welding may occur within engagement region 50 of orthopedic assembly 10, which includes both head 42 of locking screw 16 and wall 24 of plate 14. Specifically, galling or cold welding may occur between male thread 44 on head 42 of locking screw 16 and female thread 26 on wall 24 of plate 14. This galling or cold welding may make post-operative removal of orthopedic assembly 10 difficult. For example, if a surgeon tries to unscrew locking screw 16, it may become stripped or may even break apart. Should locking screw 16 become damaged, the surgeon may be forced to cut plate 14 apart and drill around locking screw 16 to remove orthopedic assembly 10.

Referring to FIG. 2, to minimize galling or cold welding within engagement region 50 of orthopedic assembly 10, coating 48 may be applied to at least one surface of engagement region 50. For example, coating 48 may be applied to head 42 of locking screw 16 or wall 24 of plate 14. To further minimize galling or cold welding within engagement region 50 of orthopedic assembly 10, coating 48 may be applied to both engaging surfaces of engagement region 50. In other words, coating 48 may be applied to both head 42 of locking screw 16 and wall 24 of plate 14 (as shown on the left side of FIG. 2). To avoid having to mask portions of plate 14 and locking screw 16 outside of engagement region 50 during the coating process, coating 48 may be applied to entire plate 14 and entire locking screw 16 (as shown on the right side of FIG. 2).

According to an exemplary embodiment of the present invention, coating 48 may be an anodized coating, such as a Type II anodized coating. Unlike a metal plate or other distinct coating layer which is deposited onto a metallic substrate, an anodized coating forms integrally with the metallic substrate by converting an exterior layer of the metallic substrate to its oxidized form. For example, if the metallic substrate is constructed of titanium, the anodized coating includes titanium oxide. Therefore, unlike plated parts, anodized parts may have no measurable dimensional change compared to the pre-anodized metallic substrate. Type II anodic treatment of titanium and its alloys is typically performed in accordance with SAE International's Aerospace Material Specification (AMS) 2488, the disclosure of which is expressly incorporated by reference herein.

Referring still to FIG. 2, coating 48 may have a thickness of approximately 0.0001 inches, 0.0002 inches, 0.0003 inches, or more, for example. The thickness of coating 48 relative to plate 14 and locking screw 16 may be exaggerated in FIG. 2.

Coating 48 may be applied to plate 14 and locking screw 16 by any suitable method. An exemplary Type II anodic treatment process is currently generally performed by Danco Anodizing, Inc. of Warsaw, Ind. This exemplary Type II anodic treatment process is set forth below. However, specific process parameters, such as the concentration of an electrolytic solution, the acidity of the electrolytic solution, the temperature of the electrolytic solution, the voltage supplied to the electrolytic solution, and the build-up layer removal technique, may vary.

First, plate 14 and/or locking screw 16 may be cleaned. For example, the components may be soaked in a hot cleaner or in a solvent bath, such as an alkaline solvent bath.

Next, plate 14 and/or locking screw 16 may be immersed in an electrolytic solution. Per the AMS 2488D specification requirements, the electrolytic solution is provided at a pH 13 or higher. A direct current is then passed from a power supply through the solution, with the power supply serving as the cathode (the negative electrode) and plate 14 and/or locking screw 16 serving as the anode (the positive electrode). Hydrogen in the solution builds up at the negative power supply, and oxygen in the solution builds up at the positive plate 14 and/or locking screw 16, which causes the surface of plate 14 and/or locking screw 16 to oxidize. The process initially forms a penetrated, hard, conversion layer and an external, soft, build-up layer.

Finally, to achieve a Type II anodized coating, the soft, build-up layer may be removed from plate 14 and/or locking screw 16. The build-up layer may be removed by immersion in an ultrasonic cleaning tank, rubbing with 0000 steel wool, or light glass-beading using a fine mesh at low pressure, for example. Removing the build-up layer exposes the hard, semi-gloss conversion layer beneath.

Advantageously, coating 48 may minimize galling or cold welding within engagement region 50 of orthopedic assembly 10. For example, the torque required to immediately separate a coated locking screw 16 from a coated plate 14 was approximately 20% to 35% less than the torque required to immediately separate an uncoated locking screw 16 from an uncoated plate 14. As another example, the torque required to immediately separate a coated locking screw 16 from a coated plate 14 was approximately 10% to 20% less than the torque required to immediately separate a coated locking screw 16 from an uncoated plate 14. Also advantageously, coating 48 may increase the mechanical strength of plate 14 and locking screw 16. In addition, coating 48 may generate a color that distinguishes plate 14 and locking screw 16 constructed of titanium, for example, from devices constructed of stainless steel.

### EXAMPLES

### 1. Example 1

### Removal Torque vs. Seating Torque

Combinations of various locking screws and plates were prepared as set forth in Table 1 below. Each locking screw was 40 mm in total length and 3.5 mm in diameter. Each plate included ten bores, each bore having dual locking holes. Three to five locking screws were tested per plate in each category, each locking screw being assigned to a different locking hole of the plate. Anodized samples were Type II anodized according to AMS 2488D or blue anodized according to DPP5358 Rev. 001024 by Danco Anodizing, Inc. of Warsaw, Ind.

TABLE 1

| | Plate | | Locking Screws | |
|---|---|---|---|---|
| | Substrate | Coating | Substrate | Coating |
| | Material | Type | Material | Type |
| 1 | Ti—6A1—4V Alloy | None | Ti—6A1—4V Alloy | Blue |
| | | | | Anodized |
| 2 | Ti—6A1—4V Alloy | None | Ti—6A1—4V Alloy | Type II |
| | | | | Anodized |
| 3 | Ti—6A1—4V Alloy | Type II | Ti—6A1—4V Alloy | Blue |
| | | Anodized | | Anodized |
| 4 | Ti—6A1—4V Alloy | Type II | Ti—6A1—4V Alloy | Type II |
| | | Anodized | | Anodized |
| 5 | Ti—15Mo Alloy | Type II | Ti—6A1—4V Alloy | Type II |
| | | Anodized | | Anodized |

Each of the five locking screw and plate combinations set forth in Table 1 above was tested according to the following procedure. A hex driver mated to a calibrated digital torque wrench was used to seat and remove the locking screws from the plates while the plates were held in a vise with the locking holes extending vertically.

First, a locking screw was seated into a hole of the plate at a torsional seating load of approximately 1.5 N-m. The locking screw was immediately removed from the plate by applying a torsional load in the opposite direction. The torsional removal load required to remove the locking screw was measured and recorded. The locking screw was then examined for signs of damage.

Next, the locking screw was seated into the same hole of the plate at a torsional seating load of approximately 2.0 N-m. Again, the locking screw was immediately removed, the torsional removal load was measured, and the locking screw was examined.

The locking screw was repeatedly seated into and removed from the same hole of the plate with increasing torsional seating loads until the hex drive sheared, which precluded further testing. The torsional seating load increased incrementally by 0.5 N-m. A maximum torsional seating load of 5.5 N-m was reached in one case before the hex drive sheared.

A new locking screw was then selected and was repeatedly seated into and removed from a new hole in the locking plate.

The test results are set forth graphically in FIG. 5. The graph represents the required removal torque versus seating torque. The dotted line represents a 1:1 correlation between removal torque and seating torque.

Referring to FIG. 5, the highest removal torques were measured when neither the locking screw nor the plate were Type II anodized (Category 1). The removal torque decreased when at least one of the locking screw and the plate was Type II anodized. The lowest removal torques were measured when both the locking screw and the plate were Type II anodized (Category 4 and Category 5). Within this group, lower removal torques were measured for a Type II anodized Ti-15Mo alloy plate than for a Type II anodized Tivanium™ Ti-6Al-4V alloy plate.

Referring still to FIG. 5, for Type II anodized plates, the difference between seating torque and removal torque tended to increase as seating torque increased (Category 3, Category 4, and Category 5).

Referring to the dotted line of FIG. 5, when the plate itself was not anodized (Category 1 and Category 2), more torque was required to remove the locking screws than was used to seat the locking screws. When the plate itself was anodized, the torque required to remove the locking screws was less than the torque used to seat the locking screws (Category 3, Category 4, and Category 5).

### 2. Example 2

### Removal Torque After Fatigue Testing

Referring back to Table 1 above, two Category 4 assemblies were prepared. Each assembly included four locking screws and a plate. A hex driver mated to a calibrated digital torque wrench was used to seat the locking screws at a torsional seating load of approximately 2.5 N-m. The assemblies were subjected to fatigue tests that occurred in Ringer's solution at 37° C. and that continued for 2 million cycles. Following fatigue testing, the locking screws were removed with the calibrated torque wrench. The torsional load required for removal was measured and recorded. The results are provided in Table 2 below.

TABLE 2

| Assembly | Screw | Torsional Removal Load |
|---|---|---|
| Number | Number | (N-m) |
| 1 | 1 | 1.71 |
| | 2 | 1.81 |
| | 3 | 1.65 |
| | 4 | 1.56 |
| 2 | 1 | 1.81 |
| | 2 | 1.39 |
| | 3 | 1.62 |
| | 4 | 1.50 |

Even after fatigue testing, the torsional load required to remove the Tivanium™ Ti-6Al-4V alloy Type II anodized locking screws from the Tivanium™ Ti-6Al-4V alloy Type II anodized plates was less than the torsional seating load of 2.5 N-m. In fact, the average torsional removal load was 1.63 N-m, which represents about a 35% reduction in torque for removal compared to seating.

While this invention has been described as having preferred designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. An orthopedic assembly comprising:
a screw having a distal end and a proximal end, the screw comprising:
a shaft;
a first thread that extends from the shaft and wraps helically around the shaft to contact a bone;
a head located at the proximal end of the screw that extends radially outwardly beyond the shaft, the head comprising an anodized coating; and
a second thread that extends from the head and wraps helically around the head; and
a plate comprising:
a bone-contacting surface configured to rest against the bone;
a wall defining a bore in the plate, the bore sized to receive the screw and comprising an anodized coating; and
a third thread that extends from the wall of the plate to interlock with the second thread of the screw.

2. The orthopedic assembly of claim 1, wherein the anodized coatings are Type II anodized coatings.

3. The orthopedic assembly of claim 1, wherein the anodized coatings comprise hard, semi-gloss, oxidized layers.

4. The orthopedic assembly of claim 1, wherein the anodized coating of the screw extends across the shaft of the screw.

5. The orthopedic assembly of claim 1, wherein the orthopedic assembly is constructed of at least one of titanium, a titanium alloy, tantalum, a tantalum alloy, zirconium, and a zirconium alloy.

6. The orthopedic assembly of claim 1, wherein the anodized coatings have a thickness less than approximately 0.0003 inches.

7. The orthopedic assembly of claim 1, wherein the anodized coatings have a thickness greater than approximately 0.0001 inches.

8. An orthopedic assembly comprising:
a screw comprising a first engagement surface and a threaded shaft configured to contact a bone;
a fixation device comprising a bone-contacting surface and a second engagement surface, the bone-contacting surface configured to contact the bone, the second engagement surface defining a bore that is sized to receive the screw such that the second engagement surface frictionally engages the first engagement surface of the screw, both the first engagement surface and the second engagement surface comprising anodized coatings.

9. The orthopedic assembly of claim 8, wherein the anodized coatings are Type II anodized coatings.

10. The orthopedic assembly of claim 8, wherein the screw comprises a head that extends radially outwardly beyond the threaded shaft to define the first engagement surface.

11. The orthopedic assembly of claim 8, wherein the first engagement surface of the screw comprises a first thread, and the second engagement surface of the fixation device comprises a second thread configured to interlock with the first thread.

12. The orthopedic assembly of claim 8, wherein the anodized coating extends across the threaded shaft of the screw.

13. The orthopedic assembly of claim 8, wherein the anodized coating extends across the bone-contacting surface of the fixation device.

14. The orthopedic assembly of claim 8, wherein the orthopedic assembly is constructed of at least one of titanium, a titanium alloy, tantalum, a tantalum alloy, zirconium, and a zirconium alloy.

15. The orthopedic assembly of claim 8, wherein the anodized coating has a thickness less than approximately 0.0003 inches.

16. The orthopedic assembly of claim 8, wherein the anodized coating has a thickness greater than approximately 0.0001 inches.

17. The orthopedic assembly of claim 8, wherein the fixation device comprises a plate configured to rest against the bone.

18. A method of securing an orthopedic device to an anatomical structure comprising the steps of:
providing an orthopedic assembly comprising:
a screw having a first engagement surface and a threaded shaft configured to contact a bone; and
a fixation device having a bone-contacting surface and a second engagement surface, the second engagement surface defining a bore, both the first engagement surface of the screw and the second engagement surface of the fixation device comprising anodized coatings;
placing the bone-contacting surface of the fixation device in contact with the bone;
inserting the screw through the bore of the fixation device;
driving the threaded shaft of the screw into the bone; and
frictionally engaging the first engagement surface of the screw with the second engagement surface of the fixation device.

19. The method of claim 18, further comprising the steps of:
immersing the screw and the fixation device in an electrolytic solution; and
passing a current through the solution to oxidize the screw and the fixation device, thereby creating anodized coatings on the screw and the fixation device.

20. The method of claim 19, further comprising the step of removing a soft build-up layer to expose a hard conversion layer beneath.

21. The method of claim 18, wherein the fixation device comprises a plate and the step of placing the bone-contacting surface of the fixation device in contact with the bone comprises resting the plate against the bone.

22. The method of claim 18, wherein the step of frictionally engaging the first engagement surface of the screw with the second engagement surface of the fixation device comprises screwing a threaded head of the screw into a threaded bore of the fixation device.
